(19) United States
(12) Patent Application Publication
(10) Pub. No.: US 2019/0192208 A1
(43) Pub. Date: Jun. 27, 2019

(54) REMOTE CONTROL ASSEMBLY FOR CATHETER SYSTEM

(71) Applicant: Cryterion Medical, Inc., Carlsbad, CA (US)

(72) Inventor: Chadi Harmouche, Saint-Laurent (CA)

(21) Appl. No.: 16/227,173

(22) Filed: Dec. 20, 2018

Related U.S. Application Data
(60) Provisional application No. 62/610,336, filed on Dec. 26, 2017.

(51) Int. Cl. A61B 18/02

(52) U.S. Cl. CPC A61B 18/02; A61B 2018/00744

(57) ABSTRACT

A remote control system for an intravascular catheter system includes a controller, a transmitter that wirelessly communicates transmitter output, and a receiver that receives the transmitter output. The receiver communicates the transmitter output to the controller to control at least a portion of the intravascular catheter system. The transmitter can include a transmitter controller and/or a transmitter interface. The transmitter controller can download, process and/or store sensor output wirelessly received from a control system. The transmitter interface can display catheter system settings and/or mirror a graphical display, and may include a touch screen. The transmitter can also wirelessly connect to technical support and/or a hospital information system. The transmitter can also wirelessly communicate transmitter output to initiate and/or terminate at least one stage of an ablation procedure.

### CROSS-REFERENCE TO RELATED APPLICATION

This application claims priority to U.S. Provisional Application Ser. No. 62/610,336 filed on Dec. 26, 2017 and entitled “REMOTE CONTROL ASSEMBLY FOR CATHETER SYSTEM”. As far as permitted, the contents of U.S. Provisional Application Ser. No. 62/610,336 are incorporated in their entirety herein by reference.

### TECHNICAL FIELD

The present disclosure relates to medical devices and methods for treating cardiac arrhythmias. More specifically, the disclosure relates to devices and methods for cardiac cryoablation.

### BACKGROUND

Cardiac arrhythmias involve an abnormality in the electrical conduction of the heart and are a leading cause of stroke, heart disease, and sudden cardiac death. Treatment options for patients with arrhythmias include medications, implantable devices, and catheter ablation of cardiac tissue.

Catheter ablation involves delivering ablative energy to tissue inside the heart to block aberrant electrical activity from depolarizing heart muscle cells out of synchrony with the heart's normal conduction pattern. The procedure is performed by positioning a portion of an energy delivery catheter adjacent to diseased or targeted tissue in the heart. The energy delivery component of the system is typically at or near a most distal (farthest from the user) portion of the catheter, and often at a tip of the device. Various forms of energy are used to ablate diseased heart tissue. These can include radio frequency (RF), ultrasound and laser energy, to name a few. One form of energy that is used to ablate diseased heart tissue includes cryogenics (also referred to herein as “cryoablation”). During an ablation procedure, with the aid of a guidewire, the distal tip of the catheter is positioned adjacent to diseased or targeted tissue, at which time the cryogenic energy can be delivered to create tissue necrosis, rendering the ablated tissue incapable of conducting electrical signals.

Atrial fibrillation is one of the most common arrhythmias treated using cryoablation. In the earliest stages of the disease, paroxysmal atrial fibrillation, the treatment strategy involves isolating the pulmonary veins from the left atrial chamber, a procedure that removes unusual electrical conductivity in the pulmonary vein. Recently, the use of techniques known as “balloon cryotherapy” catheter procedures to treat atrial fibrillation have increased. In part, this stems from ease of use, shorter procedure times and improved patient outcomes. During the balloon cryotherapy procedure, a refrigerant or cryogenic fluid (such as nitrous oxide, or any other suitable fluid) is delivered under pressure to an interior of one or more inflatable balloons which are positioned adjacent to or against the targeted cardiac tissue. Using this method, the extremely frigid cryogenic fluid causes necrosis of the targeted cardiac tissue, thereby rendering the ablated tissue incapable of conducting unwanted electrical signals.

Ablation procedures generally require the use of multiple hand-controlled structures or devices. For example, a control console may often include various structures or devices, including a graphical display, which may require the user's manual control, guidance and/or input. More specifically, the control console may be handled or used by a user, an operator or another suitable health care physician or technician (hereinafter collectively referred to as “user”), which can lead to a relatively non-sterile environment during an ablation procedure. There is a continuing need to improve the operability of cryogenic ablation systems.

### SUMMARY

The present disclosure is direct toward a remote control system for an intravascular catheter system. The remote control system includes a controller, a transmitter that wirelessly communicates transmitter output, and a receiver that receives the transmitter output. The receiver communicates the transmitter output to the controller to control at least a portion of the intravascular catheter system.

In various embodiments, the transmitter can include a transmitter interface. In one embodiment, the transmitter interface can include a touch screen. In another embodiment, the transmitter interface can use at least one of audio data and visual data. The audio data can include at least one of real time video, screen sharing and messaging. Additionally, the visual data can include at least one of real time video, screen sharing and messaging.

In certain embodiments, the intravascular catheter system can include a graphical display. The transmitter interface can be configured to selectively mirror the graphical display. In other embodiments, the intravascular catheter system can include catheter system settings. In such embodiments, the transmitter interface can selectively display the catheter system settings.

In various embodiments, the transmitter can include a transmitter controller. In some embodiments, the intravascular catheter system can include a control system that communicates system output. The control system can be wirelessly connected to the transmitter controller such that the transmitter controller can wirelessly receive the sensor output. The transmitter controller can download, process and/or store the sensor output. Additionally, in certain embodiments, the sensor output can include ablation procedure information. In such embodiments, the transmitter controller can wirelessly download and/or store the ablation procedure information.

In one embodiment, the transmitter can wirelessly connect to technical support. In another embodiment, the transmitter can wirelessly connect to a hospital information system.

In some embodiments, the intravascular catheter system is configured to control at least one stage of an ablation procedure. The ablation procedure can include at least one of an inflation stage, an ablation stage, a thawing stage and a time to isolation. The transmitter can wirelessly communicate transmitter output to initiate and/or terminate at least one stage of the ablation procedure, which may include the inflation stage, the ablation stage, the thawing stage and/or a calculation of the time to isolation.

In various embodiments, the transmitter can wirelessly communicate transmitter output to modify the catheter system settings.

In other embodiments, the transmitter can wirelessly communicate transmitter output to deactivate and/or activate the remote control system.

In certain embodiments, the intravascular catheter system can include a control console. In one embodiment, the transmitter is positioned away from the control console. In another embodiment, the controller is positioned within the control console.

The present disclosure is further directed toward a remote control system for an intravascular catheter system. The remote control system can include a controller, a transmitter that wirelessly communicates transmitter output, and a receiver that receives the transmitter output. The transmitter includes a transmitter interface and a transmitter controller. The receiver communicates the transmitter output to the controller to control at least a portion of the intravascular catheter system.

In one embodiment, the transmitter interface can include a touch screen. In another embodiment, the transmitter interface can use at least one of audio data and visual data.

In certain embodiments, the intravascular catheter system can include a graphical display. The transmitter interface can be configured to selectively mirror the graphical display. In other embodiments, the intravascular catheter system can include catheter system settings. In these embodiments, the transmitter interface can selectively display the catheter system settings.

In some embodiments, the intravascular catheter system can include a control system that communicates system output. The control system can be wirelessly connected to the transmitter controller such that the transmitter controller can wirelessly receive the sensor output. The transmitter controller can download, process and/or store the sensor output. Additionally, in certain embodiments, the sensor output can include ablation procedure information. In such embodiments, the transmitter controller can wirelessly download and/or store the ablation procedure information.

In one embodiment, the transmitter can wirelessly connect to technical support. In another embodiment, the transmitter can wirelessly connect to a hospital information system.

In some embodiments, the intravascular catheter system is configured to control at least one stage of an ablation procedure. The ablation procedure can include at least one of an inflation stage, an ablation stage, a thawing stage and a time to isolation. The transmitter can wirelessly communicate transmitter output to initiate and/or terminate at least one stage of the ablation procedure, which may include the inflation stage, the ablation stage, the thawing stage and/or a calculation of the time to isolation.

In various embodiments, the transmitter can wirelessly communicate transmitter output to modify the catheter system settings.

In other embodiments, the transmitter can wirelessly communicate transmitter output to deactivate and/or activate the remote control system.

In certain embodiments, the intravascular catheter system can include a control console. In one embodiment, the transmitter is positioned away from the control console. In another embodiment, the controller is positioned within the control console.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic side view illustration of a patient, a user and an embodiment of an intravascular catheter system having features of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

Embodiments of the present disclosure are described herein in the context of a remote control system for an intravascular catheter system. Those of ordinary skill in the art will realize that the following detailed description of the present disclosure is illustrative only and is not intended to be in any way limiting. Other embodiments of the present disclosure will readily suggest themselves to such skilled persons having the benefit of this disclosure. Reference will now be made in detail to implementations of the present disclosure as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

Although the disclosure provided herein focuses mainly on cryogenics, it is understood that various other forms of energy can be used to ablate diseased heart tissue. These can include radio frequency (RF), ultrasound, pulsed DC electric fields and laser energy, as non-exclusive examples. The present disclosure is intended to be effective with any or all of these and other forms of energy.

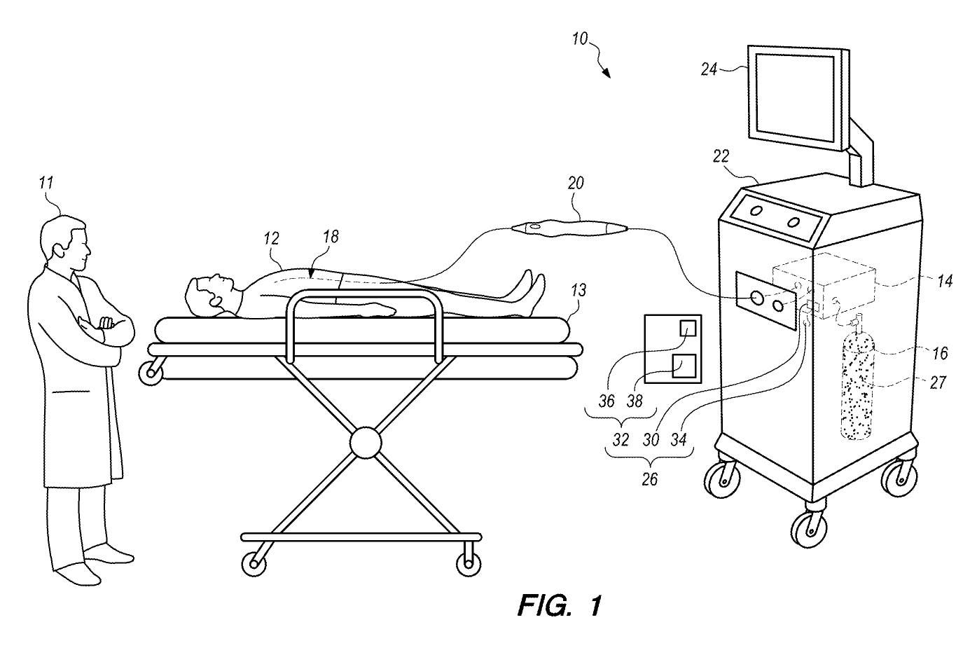

FIG. 1 is a side view illustration of one embodiment of an intravascular catheter system 10 (also sometimes referred to herein as a “catheter system”) for use by a user 11, such as a health care professional, with a patient 12, which can be a human being or an animal. In this embodiment, the user 11 operates and/or controls the catheter system 10 to perform an ablation procedure on the patient 12. While FIG. 1 shows only one user 11, it is understood that a plurality of different users 11 can operate or assist in the operation and/or control of the catheter system 10 at the same or at different times throughout the ablation procedure. In other words, the user 11 illustrated in FIG. 1 can represent any number of different users 11, i.e., a first user, a second user, etc. Further, it is understood that while specific reference is made to the user 11 as a healthcare professional, healthcare professional can include a physician, a physician's assistant, a technician, a nurse and/or any other suitable person and/or individual.

In the embodiment illustrated in FIG. 1, the patient 12 is positioned on a gurney 13. However, it is understood that the patient 12 can be positioned on any suitable surface, such as a table or a bed, as non-exclusive examples.

Although the catheter system 10 is specifically described herein with respect to the intravascular catheter system, it is understood and appreciated that other types of catheter systems and/or ablation systems can equally benefit by the teachings provided herein. For example, in certain non-exclusive alternative embodiments, the present disclosure can be equally applicable for use with any suitable types of ablation systems and/or any suitable types of catheter systems. Thus, the specific reference herein to use as part of the intravascular catheter system is not intended to be limiting in any manner.

The design of the catheter system 10 can be varied. In various embodiments, the catheter system 10 may include various controls, features and/or components to operate and/or control the catheter system 10. In addition, the catheter system 10 can include various settings, preferences, values and/or thresholds (sometimes referred to herein as “catheter system settings”). As certain non-exclusive examples, the catheter system settings may include ablation timers, safety alerts, volume level, etc. The catheter system settings may be specific to the user 11 and/or the ablation procedure to be performed. In certain embodiments, the catheter system settings can be updated and/or modified at any time. In alternative embodiments, the catheter system settings may be preprogrammed as part of the catheter system 10.

In the embodiment illustrated in FIG. 1, the catheter system 10 can include one or more of a control system 14, a fluid source 16, a balloon catheter 18, a handle assembly 20, a control console 22, a graphical display 24 (also sometimes referred to as a graphical user interface or “GUI”) and a remote control system 26. It is understood that although FIG. 1 illustrates the structures of the catheter system 10 in a particular position, sequence and/or order, these structures can be located in any suitably different position, sequence and/or order than that illustrated in FIG. 1. It is also understood that the catheter system 10 can include fewer or additional components than those specifically illustrated and described herein.

In the embodiment illustrated in FIG. 1, the user 11 operates and/or controls the catheter system 10 to perform the ablation procedure on the patient 12. Each ablation procedure can include one or more stages, such as: (i) an inflation stage, (ii) an ablation stage, (iii) a time to isolation; and (iv) a thawing stage, as non-exclusive examples. Alternatively, the ablation procedure may also include other stages not specifically mentioned herein.

As utilized herein, the “inflation stage” refers generally to the portion of the ablation procedure, wherein the cryogenic fluid 27 is being delivered from the fluid source 16 to the balloon catheter 18 at a flow rate that does not cause tissue necrosis. More specifically, the cryogenic fluid 27 is being delivered to the inflatable balloon of the balloon catheter 18. During the inflation stage, the user 11 may adjust and/or position the balloon catheter 18 within the body of the patient 12 to achieve positioning of the inflatable balloon adjacent to a targeted tissue of the patient 12. The targeted tissue can include at least a portion of heart tissue of the patient 12 that is to be treated by the catheter system 210, such as an ostium of a pulmonary vein, for example. Once positioned adjacent to the targeted tissue and the pulmonary vein is occluded, ablation of the targeted tissue may be initiated.

The “ablation stage” refers generally to the cryogenic fluid 27 being delivered from the fluid source 16 to the inflatable balloon of the balloon catheter 18 at a flow rate to create tissue necrosis. Tissue necrosis has the effect of rendering targeted tissue incapable of conducting cardiac electrical signals. During ablation of the targeted tissue, the inflatable balloon of the balloon catheter 18 is positioned adjacent to targeted tissue, with the pulmonary vein being occluded.

The “time to isolation” or “time to effect” refers to the moment when cardiac electrical signals during the ablation procedure are lost or “isolated” due to tissue ablation. It is appreciated that the time to isolation is a variable that is determined only through the process of the ablation procedure, and potentially may not actually be achieved in any given ablation procedure. As such, although the ablation procedure can be said to include a time to isolation, it is understood that the specific time to isolation for any given ablation procedure is actually unknown and only a potentiality until it happens (if it does at all) during the ablation procedure. One representative example of time to isolation would be when signals from a left atrium no longer appear in the pulmonary vein due to a circumferential lesion.

Additionally, the “thawing stage” refers generally to the stage of the ablation procedure, wherein targeted tissue of the patient 12 that has been ablated is allowed to thaw to a certain temperature and/or for a certain period of time. The thawing stage can be temperature based, time based, or both. Temperature based means that the ablated heart tissue is allowed to thaw to a certain temperature. Time based means the ablated heart tissue is allowed to thaw for a certain period of time. The temperature and period of time can vary depending on the patient 12 and/or any other ablation parameters. During the thawing stage of the targeted tissue of the patient 12, the cryogenic fluid 27 may be delivered from the fluid source 16 to the inflatable balloon of the balloon catheter 18 and/or retrieved from the inflatable balloon of the balloon catheter 18, but at a flow rate sufficient to maintain the inflatable balloon at least partially or substantially inflated to prevent the balloon catheter 18 from falling out of position and/or to reduce the likelihood of tissue damage to the patient 12.

In various embodiments, the control system 14 is configured to monitor and control the various processes of the ablation procedure. More specifically, the control system 14 can monitor and control release and/or retrieval of the cryogenic fluid 27 to and/or from the balloon catheter 18. The control system 14 can also control various structures that are responsible for maintaining or adjusting a flow rate and/or a pressure of the cryogenic fluid 27 that is released to the balloon catheter 18 during the ablation procedure. In such embodiments, the catheter system 10 delivers ablative energy in the form of cryogenic fluid 27 to cardiac tissue of the patient 12 to create tissue necrosis, rendering the ablated tissue incapable of conducting electrical signals. Additionally, in various embodiments, the control system 14 can control activation and/or deactivation of one or more other processes of the balloon catheter 18. Further, or in the alternative, the control system 14 can receive electrical signals, data and/or other information (also sometimes referred to as “sensor output”) from various structures within the catheter system 10. In various embodiments, the control system 14 and/or the GUI 24 can be electrically connected and/or coupled to each other. In some embodiments, the control system 14 can assimilate and/or integrate the sensor output and/or any other data or information received from any structure within the catheter system 10. Additionally, or in the alternative, the control system 14 can control positioning of portions of the balloon catheter 18 within a circulatory system (not shown) (also sometimes referred to herein as the “body”) of the patient 12, and/or can control any other suitable functions of the balloon catheter 18.

Additionally, as provided herein, the control system 14 can also wirelessly receive data, electronic or other signals and/or information from the remote control system 26 (hereinafter sometimes referred to herein as “transmitter output”). For example, in certain embodiments, the transmitter output can function initiate and/or terminate any stage of the ablation procedure, including the inflation stage, the ablation stage, and/or the thawing stage, for example. In other embodiments, the transmitter output can function to initiate the measurement and/or calculation of certain stages of the ablation procedure, which may include a time to isolation. In still other embodiments, the transmitter output can function to initiate and/or terminate timers. In yet other embodiments, the transmitter output can function to activate and/or deactivate the remote control system 26. Additionally, and/or alternatively, the transmitter output can include other data, signals and/or information corresponding to other suitable functions of the catheter system 10 that may be wirelessly controlled by the user 11. In some embodiments, the control system 14 can assimilate and/or integrate the transmitter output received from the remote control system 26.

The fluid source 16 (also sometimes referred to as “fluid container 16”) can include one or more fluid container(s) 16. It is understood that while one fluid container 16 is illustrated in FIG. 1, any suitable number of fluid containers 16 may be used. The fluid container(s) 16 can be of any suitable size, shape and/or design. The fluid container(s) 16 contains the cryogenic fluid 27, which is delivered to the balloon catheter 18 with or without input from the control system 14 during the ablation procedure. Once the ablation procedure has initiated, the cryogenic fluid 27 can be injected or delivered and the resulting gas, after a phase change, can be retrieved from the balloon catheter 18, and can either be vented or otherwise discarded as exhaust (not shown). More specifically, the cryogenic fluid 27 delivered to and/or removed from the balloon catheter 18 can include a flow rate that varies. Additionally, the type of cryogenic fluid 27 that is used during the ablation procedure can vary. In one non-exclusive embodiment, the cryogenic fluid 27 can include liquid nitrous oxide. In another non-exclusive embodiment, the cryogenic fluid 27 can include liquid nitrogen. However, any other suitable cryogenic fluid 27 can be used.

The design of the balloon catheter 18 can be varied to suit the design requirements of the catheter system 10. As shown, the balloon catheter 18 is inserted into the body of the patient 12 during the ablation procedure. In one embodiment, the balloon catheter 18 can be positioned within the body of the patient 12 using the control system 14. Stated in another manner, the control system 14 can control positioning of the balloon catheter 18 within the body of the patient 12. Alternatively, the balloon catheter 18 can be manually positioned within the body of the patient 12 by the user 11. In certain embodiments, the balloon catheter 18 is positioned within the body of the patient 12 utilizing at least a portion of the sensor output that is received from the balloon catheter 18. For example, in various embodiments, the sensor output is received by the control system 14, which can then provide the user 11 with information regarding the positioning of the balloon catheter 18. Based at least partially on the sensor output feedback received by the control system 14, the user 11 can adjust the positioning of the balloon catheter 18 within the body of the patient 12 to ensure that the balloon catheter 18 is properly positioned relative to targeted cardiac tissue. While specific reference is made herein to the balloon catheter 18, as noted above, it is understood that any suitable type of medical device and/or catheter may be used.

The handle assembly 20 is handled and used by the user 11 to operate, position and control the balloon catheter 18. The design and specific features of the handle assembly 20 can vary to suit the design requirements of the catheter system 10. In the embodiment illustrated in FIG. 1, the handle assembly 20 is separate from, but in electrical and/or fluid communication with the control system 14, the fluid container 16 and the GUI 24. In some embodiments, the handle assembly 20 can integrate and/or include at least a portion of the control system 14 within an interior of the handle assembly 20. In one embodiment, the user 11 can steer and/or navigate the balloon catheter 18 by utilizing the handle assembly 20. It is understood that the handle assembly 20 can include fewer or additional components than those specifically illustrated and described herein.

In the embodiment illustrated in FIG. 1, the control console 22 includes at least a portion of the control system 14, the fluid container 16 and/or the GUI 24. However, in alternative embodiments, the control console 22 can contain additional structures not shown or described herein. Still alternatively, the control console 22 may not include various structures that are illustrated within the control console 22 in FIG. 1. For example, in certain non-exclusive alternative embodiments, the control console 22 does not include the GUI 24.

In various embodiments, the GUI 24 is electrically connected to the control system 14. Additionally, the GUI 24 provides the user 11 of the catheter system 10 with information that can be used before, during and after the ablation procedure. For example, the GUI 24 can provide the user 11 with information based on the sensor output, and any other relevant information that can be used before, during and after the ablation procedure. The specifics of the GUI 24 can vary depending upon the design requirements of the catheter system 10, or the specific needs, specifications and/or desires of the user 11.

In one embodiment, the GUI 24 can provide static visual data and/or information to the user 11. In addition, or in the alternative, the GUI 24 can provide dynamic visual data and/or information to the user 11, such as video data or any other data that changes over time, e.g., during the ablation procedure. Further, in various embodiments, the GUI 24 can include one or more colors, different sizes, varying brightness, etc., that may act as alerts to the user 11. Additionally, or in the alternative, the GUI 24 can provide audio data or information to the user 11.

The remote control system 26 allows the user 11 to wirelessly operate and/or control at least a portion of the catheter system 10, which may also include during the ablation procedure. The portion of the catheter system 10 can include any component or structure described herein, including any function of such components or structures. Additionally, as used herein, the term “wirelessly” refers to any form of wireless technology that allows the user 11 to operate and/or control the catheter system 10 wirelessly or remotely from the patient 12. As certain non-exclusive examples, wireless technology can include infrared light, radio frequency, Bluetooth connectivity, voice control and/or Wi-Fi. Alternatively, wireless technology can include any other suitable technology that allows the user 11 to remotely operate and/or control the catheter system 10. In some embodiments, the user 11 may wirelessly operate and/or control the catheter system 10 from a distance, such as from a control room, for example.

In certain embodiments, the remote control system 26 can be wirelessly connected to the control system 14 and the GUI 24. Accordingly, any transmitter output that is transmitted or sent by the remote control system 26 can be used by the control system 14 to operate, control, update and/or modify portions of the catheter system 10, including catheter system settings, as one non-exclusive example. Additionally, any transmitter output and any information or data that is based on the transmitter output can be displayed in visual and/or in audio format on the GUI 24.

The design and specific features of the remote control system 26 can vary to suit the design requirements of the catheter system 10. In the embodiment illustrated in FIG. 1, the remote control system 26 can include one or more of a controller 30, a transmitter 32, and a receiver 34. It is understood that the remote control system 26 can include additional components than those specifically illustrated and described herein.

The controller 30 processes transmitter output and/or sensor output. In certain embodiments, the controller 30 can process the transmitter output to operate and/or control various portions of the catheter system 10. More specifically, in some embodiments, the controller 30 can process the transmitter output to initiate and/or terminate certain stages of the ablation procedure. For example, the controller 30 can process transmitter output to initiate and/or terminate the inflation stage, the ablation stage and/or the thawing stage, as non-exclusive examples. Alternatively, the controller 30 can process transmitter output to initiate and/or terminate a calculation of the time to isolation. Still alternatively, the controller 30 can process transmitter output to cause the catheter system 10 to perform any other suitable function.

In the embodiment illustrated in FIG. 1, the controller 30 can be integrated and/or included as part of the control system 14. In other embodiments, the controller 30 can be integrated and/or included as part of any other suitable structure within the catheter system 10, such as the control console 22, for example.

In various embodiments, the controller 30 can include at least one processor (e.g., microprocessor) that executes software and/or firmware stored in memory of the controller 30. The software/firmware code contains instructions that, when executed by the processor, cause the controller 30 to perform the functions of the control algorithm described herein. The controller 30 may alternatively include one or more application-specific integrated circuits (ASICs), field-programmable gate arrays (FPGAs), digital signal processors (DSPs), hardwired logic, or combinations thereof. The controller 30 may receive information from a plurality of system 10 components and feed the information (e.g., sensor data, signals from the transmitter 32, and user inputs from the GUI 24) into a control algorithm which determines at least one control parameter which may in part govern operation of the catheter system 10.

The transmitter 32 selectively communicates transmitter output to the controller 30. The transmitter 32 can be selectively and/or manually actuated by the user 11 via any suitable manner or method. The design of the transmitter 32 can also vary depending on the design requirements of the catheter system 10. In the embodiment illustrated in FIG. 1, the transmitter 32 includes one or more of a transmitter controller 36 and a transmitter interface 38. It is understood that the transmitter 32 can include fewer or additional components than those specifically illustrated and described herein. Alternatively, the transmitter 32 may not include various structures that are illustrated within the transmitter 32 in FIG. 1. For example, in one embodiment, the transmitter 32 may not include the transmitter interface 38. Additionally, in one embodiment, the transmitter 32 can be covered with a transparent sterile cover (not shown) such that the transmitter 32 can be operated and/or controlled in a more sterile manner. Furthermore, in various embodiments, the transmitter 32 can be positioned away from the control console 22 such that the user 11 may wirelessly operate and/or control the catheter system 10 without having to manually access the control console 22.

In certain embodiments, the transmitter controller 36 can be wirelessly connected to the control system 14. Accordingly, any sensor output that is processed and/or stored by the control system 14 can be wirelessly received and/or processed by the transmitter controller 36, and vice versa. The transmitter controller 36 can wirelessly receive or access sensor output via any suitable method. The design of the transmitter controller 36 can also vary. In the embodiment illustrated in FIG. 1, the transmitter controller 36 is integrated and/or included as part of the transmitter 32. Alternatively, the transmitter controller 36 may be integrated and/or included as part of any other suitable component or structure of the remote control system 26.

In one embodiment, the transmitter controller 36 can store the sensor output that has been wirelessly received or accessed from the control system 14. In other embodiments, the transmitter controller 36 can be configured to download and/or process the sensor output. For example, the transmitter controller 36 can process the sensor output by organizing the sensor output to generate reports. In some embodiments, the sensor output and/or reports may include any suitable data and/or information relating to the ablation procedure (sometimes referred to herein as “ablation procedure information”). As certain non-exclusive examples, the ablation procedure information can include a time to reach a target ablation temperature, a time to reach a target ablation pressure, and/or a summary that includes one or more of the following: (i) one or more ablation locations, (ii) a number of ablations at each location and (iii) a duration of each ablation at each location. Alternatively, the sensor output and/or reports may include any other suitable information that would be instructive and/or useful to the user 11. In certain embodiments, the transmitter controller 36 can be configured to allow the transmitter 32 to cause the sensor output to print, which may include printing the generated reports, for example.

Additionally, in various embodiments, the transmitter 32 can be configured to wirelessly connect to a hospital information system. The hospital information system generally refers to a central electronic information system of a hospital that secures and/or controls data and/or information of a patient 12, which may also include ablation procedure information. In certain embodiments, the controller transmitter 36 can also be configured to download and store the ablation procedure information.

In various embodiments, the transmitter interface 38 is electrically connected to the transmitter controller 36. In the embodiment illustrated in FIG. 1, the transmitter interface 38 is integrated and/or included as part of the transmitter 32. The design of the transmitter interface 38 can vary. Additionally, the transmitter interface 38 provides the user 11 of the catheter system 10 with information that can be used before, during and after the ablation procedure. For example, the transmitter interface 38 can provide the user 11 with information based on the sensor output, the transmitter output, and any other relevant information that can be used before, during and/or after the ablation procedure. The specifics of the transmitter interface 38 can vary depending upon the design requirements of the catheter system 10, or the specific needs, specifications and/or desires of the user 11.

In some embodiments, the transmitter interface 38 can display in visual and/or in audio format any sensor output, transmitter output and/or any information or data that is based on the sensor output and/or transmitter output. For example, in some embodiments, the transmitter interface 38 can selectively use or provide at least one of audio and visual data for the transmitter 32 to connect to technical support for troubleshooting. The audio and visual data can include one of real time video, screen sharing and/or messaging, as certain non-exclusive examples. More specifically, the transmitter interface 38 can provide static visual data and/or information to the user 11. In addition, or in the alternative, the transmitter interface 38 can provide dynamic visual data and/or information to the user 11, such as video data or any other data that changes over time. The transmitter interface 38 can also include one or more configurations of the visual data depending upon the needs and/or desires of the user 11. Further, in various embodiments, the transmitter interface 38 can include one or more colors, different sizes, varying brightness, etc., that may act as alerts to the user 11.

In various embodiments, the transmitter interface 38 can be configured to selectively mirror the GUI 24. As used herein, “mirror” can include the transmitter interface 38 providing a real-time visual display of information or data, including sensor output and/or transmitter output, which is displayed on the GUI 24 at any time. In other embodiments, the transmitter interface 38 can selectively display catheter system settings, which may include mirroring catheter system settings that are displayed on the GUI 24.

In certain embodiments, the transmitter interface 38 can include a touch screen. In such embodiments, the user 11 may interact with the touch screen to wirelessly operate and/or control any portion or function of the catheter system 10, which may include during the ablation procedure. The user 11 may interact with the touch screen in any suitable manner. More specifically, as one non-exclusive example, the touch screen may include a display of a plurality of visual buttons corresponding to the operation and/or control of various portions or functions of the catheter system 10, such that alternatingly touching each of the visual buttons, selectively causes the transmitter 32 to communicate transmitter output corresponding to that portion or function of the catheter system 10. Alternatively, the touch screen may include any form of display that allows the interaction of the user 11 to operate and/or control any portion or function of the catheter system 10. Additionally, and/or alternatively, in various embodiments, the user 11 may interact with the touch screen to communicate, receive or access, provide input, and/or control sensor output, transmitter output and/or any information or data that is based on the sensor output and/or transmitter output.

In some embodiments, the receiver 34 receives and/or communicates the transmitter output and/or sensor output. The design of the receiver 34 can vary. In the embodiment illustrated in FIG. 1, the receiver 34 is electrically connected to the controller 30. The receiver 34 may be connected to the controller 30 via any suitable manner. In this embodiment, once the receiver 34 receives the transmitter output, the receiver can communicate the transmitter output to the controller 30. The controller 30 may then process the transmitter output and cause at least a portion of the catheter system 10 to operate in response to the transmitter output. In other embodiments, the receiver 34 can be connected to other structures in the catheter system 10, such as the control system and/or control console 22, for example. Alternatively, the receiver 34 can be positioned, integrated and/or included as part of any other suitable structure in the catheter system 10.

It is understood that although a number of different embodiments of the remote control system 26 have been illustrated and described herein, one or more features of any one embodiment can be combined with one or more features of one or more of the other embodiments, provided that such combination satisfies the intent of the present disclosure.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

I claim:

1. A cryoablation catheter system comprising:
a cryoablation catheter including a cryoballoon configured to be advanced within a body of a patient and to deliver cryoablation energy to target tissue of a patient;
a control console operatively coupled to the cryoablation catheter and including a cryogenic fluid supply configured to supply cryogenic fluid to the cryoballoon; and
a remote control system including:
a controller configured to control one or more stages of an ablation procedure;
a transmitter configured to be located remotely from the controller and to wirelessly transmit a transmitter output; and
a receiver operatively coupled to the controller and configured to receive the transmitter output and communicate the transmitter output to the controller to cause the controller to control the at least one or more stages of the ablation procedure.

2. The cryoablation catheter system of claim 1, wherein the control console further includes a control system that is configured to wirelessly communicate sensor output to the transmitter.

3. The cryoablation catheter system of claim 2, wherein the transmitter is configured to store and process the sensor output.

4. The cryoablation catheter system of claim 3, wherein the sensor output includes ablation procedure information.

5. The cryoablation catheter system of claim 1, wherein the transmitter is configured to wirelessly communicate the transmitter output to the controller to initiate the at least one stage of the ablation procedure.

6. The cryoablation catheter system of claim 5, wherein the at least one stage of the ablation procedure includes at least one of an inflation stage, an ablation stage, a thawing stage and a time to isolation, and wherein the transmitter is configured to wirelessly communicates the transmitter output to the controller to initiate at least one of the inflation stage, the ablation stage, the thawing stage and a calculation of the time to isolation.

7. The cryoablation catheter system of claim 1, wherein the transmitter is configured to wirelessly communicate the transmitter output to the controller to terminate the at least one stage of the ablation procedure.

8. The cryoablation catheter system of claim 7. wherein the at least one stage of the ablation procedure includes at least one of an inflation stage, an ablation stage and a thawing stage, and wherein the transmitter is configured to wirelessly communicate the transmitter output to the controller to terminate at least one of the inflation stage, the ablation stage and the thawing stage.

9. The cryoablation catheter system of claim 1, wherein the transmitter is configured to wirelessly communicate the transmitter output to the controller to modify catheter system settings.

10. A cryoablation catheter system comprising:
a cryoablation catheter including a cryoballoon configured to be advanced within a body of a patient and to deliver cryoablation energy to target tissue of a patient;
a control console operatively coupled to the cryoablation catheter and including a cryogenic fluid supply configured to supply cryogenic fluid to the cryoballoon; and
a remote control system including:
a controller configured to control one or more stages of an ablation procedure;
a transmitter configured to be located remotely from the controller and to wirelessly transmit a transmitter output, the transmitter including a transmitter interface; and
a receiver operatively coupled to the controller and configured to receive the transmitter output and communicate the transmitter output to the controller to cause the controller to control the at least one or more stages of the ablation procedure.

11. The cryoablation catheter system of claim 10, wherein the controller is located within the control console.

12. The cryoablation catheter system of claim 10, wherein the transmitter interface includes a touch screen.

13. The cryoablation catheter system of claim 12, wherein the transmitter interface is configured to display at least one of audio data and visual data.

14. The cryoablation catheter system of claim 10, further comprising a graphical display, wherein the transmitter interface is configured to selectively mirror the graphical display.

15. The cryoablation catheter system of claim 14 wherein the graphical display is configured to display catheter system settings, and wherein the transmitter interface is configured to selectively display the catheter system settings.

16. A remote control system for a cryoablation catheter system including a cryoablation catheter and a control console operatively coupled to the cryoablation catheter and including a cryogenic fluid supply, the remote control system comprising:
a controller configured to control one or more stages of an ablation procedure;
a transmitter configured to be located remotely from the controller and to wirelessly transmit a transmitter output, the transmitter including a transmitter interface; and
a receiver operatively coupled to the controller and configured to receive the transmitter output and communicate the transmitter output to the controller to cause the controller to control the at least one or more stages of the ablation procedure.

17. The remote control system of claim 16, wherein the controller is located within the control console.

18. The remote control system of claim 17, wherein the transmitter interface includes a touch screen and is configured to display at least one of audio data and visual data.

19. The remote control system of claim 18, wherein the cryoablation catheter system further comprises a graphical display, and wherein the transmitter interface is configured to selectively mirror the graphical display.

20. The remote control system of claim 19, wherein the graphical display is configured to display catheter system settings, and wherein the transmitter interface is configured to selectively display the catheter system settings.
